(19) United States
(12) Patent Application Publication
(10) Pub. No.: US 2008/0097144 A1
(43) Pub. Date: Apr. 24, 2008

(54) Apparatus for collection of oocytes

(75) Inventors: Michael D. Cecchi, Madison, CT (US); David Popolizio, East Haven, CT (US)

Correspondence Address: William W. Jones, 6 Juniper Lane, Madison, CT, 06443, US

(73) Assignee: Genx International, Inc.

(21) Appl. No.: 11/491,666

(22) Filed: Jul. 24, 2006

(51) Int. Cl. A61B 17/425

(52) U.S. Cl. 600/33

(57) ABSTRACT

An oocyte aspirator assembly for use in in vitro fertilization procedures includes a vacuum source for creating a vacuum in the assembly. The assembly also includes valve mechanisms to selectively vary the intensity of the vacuum created in the assembly between at least two levels, one of which is at a first low level at which oocytes can be safely aspirated from ovarian follicles, and a second of which is at a higher level than the first low level. The assembly also includes a control which can be manually or automatically actuated to change the vacuum level in the assembly. The control can take the form of one or more foot pedals or an occlude button mounted on a casing for the assembly. The assembly also may include a third vacuum level which equals the full output of the vacuum motor.

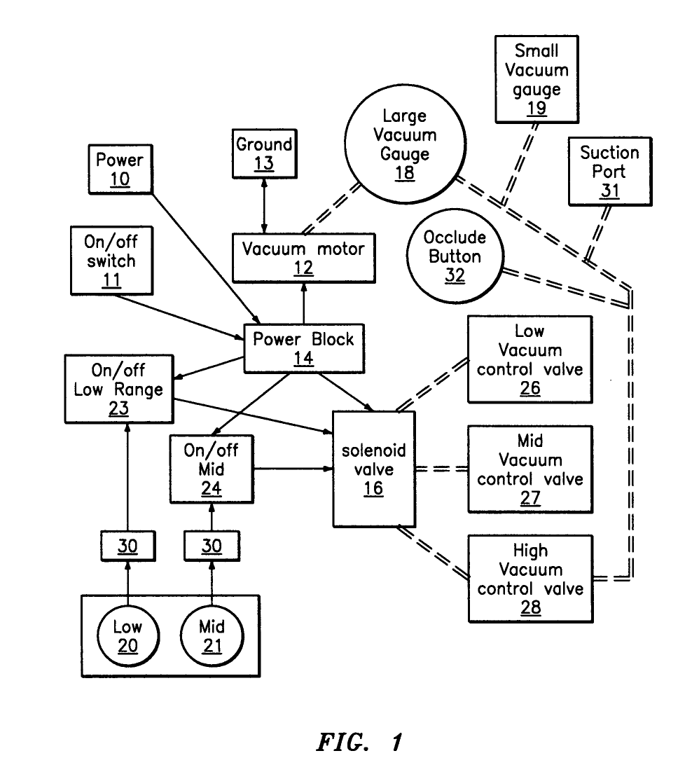

### TECHNICAL FIELD

This invention pertains to assisted reproduction techniques in general, and to methods and apparatus for oocyte collection in particular.

### BACKGROUND OF THE INVENTION

A variety of techniques have been developed to collect oocytes from ovarian follicles for the purpose of fertilization in vitro. Most of the techniques involved the insertion of an aspirating needle into an ovarian follicle. The aspirating needle is connected by tubing to a material collection trap and the collection trap, in turn, is connected to a suction source. Once the aspirating needle is inserted into the follicle, the follicle is aspirated to remove the oocyte and fluid residing within the follicle. The fluid and oocyte are collected into the collection trap. Early aspirating techniques utilized a manually operated syringe as a suction source. Syringe-type suction requires considerable technician training and technique. Even with such training and technique, it can be difficult to produce the constant level of suction that is best suited to collecting viable oocytes without damage. In addition, the length of time that syringe suction can be applied is limited by the stroke of the syringe.

Subsequent aspirating techniques utilize an electromechanical vacuum source connected to the aspirating needle and trap apparatus. The electromechanical aspirating vacuum source(s) utilze a vacuum pump having two vacuum settings. One of the vacuum settings represents a low vacuum level which may be preset by the manufacturer of the vacuum pump or may be manually set by the user by adjusting a suction control valve. The second vacuum setting is not set by the user but is instead the free flow vacuum level of the vacuum pump unit used by the manufacturer. The manufacturers utilize a vacuum motor which has a top vacuum of about 400-500 mm Hg. In order to achieve a high vacuum of the aspiration unit the manufacturers simply allow the vacuum motor within the aspiration unit to free flow to its maximum vacuum capability, without inhibiting the vacuum or using a control valve to set or control the vacuum level.

A problem with this type of aspirating vacuum source in this type of aspiration unit is that it limits the operator to one of two vacuum levels, one is settable and the other is dictated by the manufacturer by their selection of the vacuum motor used. If the operator determines that the low setting is not providing sufficient vacuum to aspirate a follicle, then he must use the higher vacuum setting. If this vacuum setting is too high, it may cause damage to the fragile oocytes being aspirated from the follicle. The inability to properly adjust the suction pressure may cause damage and may be detrimental to the viability of the oocytes. This damage may be permanent and impact the viability and survival of the embryos.

Our studies have shown that when the aspiration occurs at a low setting, this is a safe level for aspiration using a vacuum of 90 mm Hg but occasionally the needle or tubing may become clogged with tissue, blood or debris. When this happens it is necessary for the operator to increase the vacuum used to high to unclog the system. When the vacuum apparatus is increased to a high setting of between 350-500 mm Hg, as per the prior art, the oocyte may become damaged and permanently impaired. The ability to control the vacuum using a secondary level of an operator-preset vacuum rate would allow the user to aspirate at additional safe ranges of 120-130 mm Hg and 150-160 mm Hg which may overcome aspiration impediments and still allow aspiration without causing damage to the embryos.

Another problem with the current aspiration unit in use is that the operator is not able to aspirate in the low vacuum of 90 mmHg and then move to the secondary vacuum of 120 mm Hg, hands free. The operator, or physician, in most cases, has both hands in use and any incremental increase from the low vacuum to a secondary vacuum must be done manually.

Additional problems occur relating to the visibility of the pressure gauge settings. Currently the units use analog gauges to display the vacuum level, these gauges are currently printed to display 0 to 750 mmHg vacuum levels. Therefore the value of 90 mm Hg has a small display range on the pressure gauge dial and the user typically has trouble determining the range visually.

What is needed is a method and an apparatus for collecting oocytes that can repeatedly provide favorable conditions for successful oocyte collection, one that relies on less technician training and technique than is required under the prior art, and one that can be used in human and veterinarian practices.

### DISCLOSURE OF THE INVENTION

This invention relates to an improved aspirator which includes an incoming power supply, a vacuum source, air electric switches, a control panel which includes the external devices, and a suction inlet. The vacuum source is an electromechanical pump capable of producing a range of vacuum extending between zero to five hundred millimeters of mercury (0-500 mm Hg) in a one inch or smaller diameter tube for a period of time long enough to permit the completion of the aspiration procedure at hand. The vacuum source may be controlled to maintain any given selected vacuum level to within plus or minus two mm Hg at the given vacuum level. An example of an acceptable vacuum for human applications is 50 mm Hg to 150 mm Hg. The aspirator control panel includes a low vacuum selector, a mid vacuum selectors, a high vacuum selector or occlude button to attain a high vacuum, a digital or manual selector switching mechanism, and a vacuum level indicator in digital or analogue readout. The vacuum selectors are disposed between the vacuum source and the aspirating equipment, and may be a pneumatic, electromechanical or mechanical type device. The mechanical device could be a needle valve, manufactured by Pneumadine. Each vacuum selector controls the amount of vacuum drawn by the aspirator at each level and can be selectively adjusted within that range. The range of each selector may overlap the range of the adjacent selector; i.e., the low and mid vacuum selectors may have ranges that overlap each other to an extent, and the mid range and high vacuum selectors may also have ranges that overlap each other to an extent. Collectively, the low, mid and high ranges form the entire range of the aspirator. The vacuum selectors can be capable of achieving multiple mid ranges, and may have at least three distinct vacuum levels: the low, mid ranges and high ranges. The vacuum selectors may also be capable of achieving multiple mid ranges, and may have at least four distinct vacuum levels: the low, two mid ranges and the high range. The mid range setting may be the highest setting for safe aspiration of the oocyte, but not the highest vacuum available from the pump. Each vacuum selector can be preset at a particular vacuum setting selected within its range. The vacuum selector switching mechanism enables the operator to change between the different vacuum selectors, and more specifically between the preset vacuum levels of each selector. In some instances, the selector switching mechanism may be operable directly from the control panel. In other instances, the switching mechanism may be operable by a foot pedal switch connected to the control panel.

A multi-port solenoid valve or series of solenoid valves are used to make the change between the level of the vacuum, which is controlled through the foot pedal or by depressing a button on the panel of the unit. The vacuum may be changed through the use of the foot pedal by depressing a separate portion of the foot pedal which activates an air electric switch which, in turn, opens or closes the appropriate vacuum selector or selectors to achieve the desired vacuum level.

This may also be accomplished by the use of a preset or settable vacuum level controlled by an electrical signal sent from the control board to the desired air control valve, opening and dosing the valves in a specific sequence to achieve the desired vacuum level.

The vacuum levels are controlled by the use of multi-port solenoid valve or series of solenoid valves that work in various sequences to achieve the multiple desired levels of vacuum. The foot pedal may be used to turn the unit on and off, and to maintain an “on” position. The pressure switching mechanism may activate through a series of foot pedals, or by a sequence in which one pedal is depressed. The foot pedal may also work in combination with a series of buttons on the control panel to achieve the desired and various vacuum levels.

The vacuum level gauge indicator is a display device to show the operator the level of vacuum presently being drawn through the aspirating equipment. The indicator may be a digital or an analog device. The control panel further includes an indicator light for each vacuum selector to dearly identify which vacuum selector is engaged. Additional functionality can also be built into the control panel such as power switches for the vacuum source and the control panel itself.

In the operation of the aspirator of this invention, the vacuum selectors are pre adjusted to predetermined settings prior to the onset of the follicular aspiration. The predetermined settings are based on clinically developed data that organizes patients as a function of certain characteristics; e.g., age, height, weight, health, etc. The predetermined setting may also be determined by the type of oocyte to be aspirated. This variance is determined by the maturity of the oocyte, as on occasion oocytes may be aspirated at an immature growth level, due to various techniques and drug regimentations. It is our understanding that clinically developed data now suggests, or will suggest, certain trends that can be used to identify optimum vacuum settings for the collection of oocytes from any particular patient based on characteristics of the patient.

One current trend in the use of this aspiration mechanism is to aspirate oocytes at various development of the maturation within the ovum. As patients' response to fertility drugs and allergic reaction to drugs may after the physicians' need to retrieve oocytes at various stages of development. The physician may be required to retrieve immature oocytes, which are smaller in size and may be more prone to damage. Secondly, this retrieval technique at times uses a smaller 18 or 19 gauge aspiration needle when the average size used is 17 gauge. This change in the required size of the needle requires a very low setting on the aspiration unit due to the increased acceleration of fluids, along with the oocytes due to the reduced size of the needles but not of the tubing attached to the apparatus, and if a conventional setting were used, it would increase the likelihood of damage to the oocytes and reduce the likelihood of success of the procedure.

As noted above, with the use of conventional equipment, the aspirating needle may become dogged with tissue, blood or other debris. Current devices only allow the user to switch from a safe low setting of from approximately 20 mm Hg to approximately 120 mm Hg to the high setting of from 350-500 mm Hg. The current invention allows the user to move from the low setting to a higher low mid range setting of possibly 120 mm Hg to unclog the system, and if this fails to do so, the user may then move to the high, but safe, mid range of possibly 130 mm Hg to unclog the system which would still allow the oocytes to be aspirated with a safe range with less likelihood of damage.

The collection apparatus housing may contain two separate vacuum gauges. The purpose of the gauges is to allow the user to view a large gauge, with a greater surface area to allow greater separation between the levels of vacuum. This will allow the user more easily and accurately set the vacuum levels. This will also allow the user to be able to view the face of the gauge and observe the vacuum level more easily. The first gauge may only be for the safe range of the aspiration of oocytes, as described earlier. The second gauge will allow the user to observe the higher levels of the vacuum range. This will give a conventional user a certain comfort in the visualization of what they may be currently be used to and also allow the unit to be used in a different capacity, thereby not restricting its use to the lower vacuum ranges or to upper vacuum ranges.

It is, therefore, an object of the present invention to provide a safer method for aspirating oocytes from the ovum with a mechanical vacuum unit. The preferable method would be to be able to achieve multiple safe levels of a vacuum, which may be preset or adjusted by the user.

### BRIEF DESCRIPTION OF THE DRAWING

These and other objects, features and advantages of the present invention will become more apparent in light of the detailed description thereof, as illustrated in the accompanying drawing.

The sole figure shows the inner workings of one embodiment of the aspiration system and unit of this invention.

### DETAILED DISCLOSURE OF THE INVENTION

Referring now to the drawing, the aspirator system has an incoming power supply 10, which supplies power to the system through a common power block 14. The on/off switch 11 will turn the entire system on and put it in a ready mode. A vacuum motor 12 draws its power from the block 14. The vacuum motor is grounded 13. The power is activated, from the ready mode to an active mode by depressing foot pedal 20, or 21. For this example, the system has two separate foot pedal actuators 20, 21. By depressing foot pedal 20, a message will be send to activate the system. This message may be an air or electrical signal 30. By depressing the foot pedal 20, the system would be activated, and in this case would be activated to accommodate a predetermined low vacuum level. When foot pedal 20 is depressed, it activates an air/electric switch 23 and turns on the vacuum motor 12 to create a low vacuum. The switch 23 takes an air impulse and turns on an electric switch. Alternatively, the switch 23 could react to an electric impulse to switch on and off. At the same time a three-way electric solenoid valve 16 is activated, which directs the vacuum created by the vacuum motor 12 through the 3-way solenoid valve 16, then through a low level vacuum control valve 26, which may be a simple needle valve, and will allow the vacuum to be adjusted within what is considered a low range. In this example, up to between 20 and 90 mm Hg. For the example the valve has been adjusted to a vacuum of 75 mm Hg. The vacuum would be adjusted by valve 26, and would pass through vacuum gauge 18, which will give a visible reading to the user. The vacuum would then continue to the suction port 31, which in this case is mounted on the front panel of the aspirator system enclosure. The suction port is where the user would attach an aspiration device to be used.

For this example the on/off switch 11, connector(s) for the foot pedals, the low control valve 26, mid vacuum control valve 27, an occlude button 32 to achieve the high 28 vacuum, vacuum gauge 18 and suction port 31 can be mounted on the exterior of the system or on a control panel (not shown) on the exterior of the casing.

As depicted by the drawing, a second example of the system is if the user wished to achieve a safe mid vacuum range. This may be used after using a low vacuum level or initially as a mid vacuum level. The user would depress the foot pedal 21 which is the designated mid range foot pedal. When foot pedal 21 is depressed, it activates an air-electric switch 24 which activates the vacuum motor 12 to create a vacuum. At the same time the three-way solenoid valve 16 is activated, which directs the vacuum created by the vacuum motor 12, through the three-way solenoid valve 16, to the mid level vacuum control valve 27, which may be a simple needle valve, and will allow the vacuum to be adjusted within what is considered a mid range. In this example, we consider the safe range to be from 100 mm Hg to 120 mm Hg, and the control valve 27 is set to 120 mm Hg. Upon depression of the foot pedal 21, the vacuum will then go to 120 mm Hg and will pass through the rear of the vacuum gauge 18, which will give a visible reading to the user. The vacuum would then continue to the suction port 31, which in this case is mounted on the front panel of the unit.

A third example of use of the system is if the user wishes to achieve a high vacuum of over 120 mm Hg or up to the free flow of the vacuum motor 12. In this case the vacuum motor 12 has a 350 mm Hg rating in free flow. This may be after using a low or mid vacuum level. The user would depress the occlude button 32 on a control panel on the enclosure of the assembly. When this is done it closes the system and pushes the system to 350 mm Hg, or the free flow of the vacuum motor and the maximum of the pump. If the system as a vacuum control valve in 28 it may be set for a range above the setting of the second valve or in this case above 120 mm Hg, the vacuum pressure in the system would go to 120 mm Hg, and would show 120 on the vacuum gauge 18. The vacuum would then continue to the suction port 31, which in this case is mounted on the front panel of the unit.

An alternative embodiment of the invention involves the use of two separate vacuum gauges 18 and 19. Vacuum gauge 18 has a scale and a working range of 0 to 300 mm Hg, on its display face. This would be the operating range for the user. This would allow the user to more accurately set and read the various pressure levels. In this example the user would be able to read the low setting of 90 mm Hg and the mid setting of 120 mm Hg. They would be able to more accurately set the vacuum as well as read the levels during the procedures. The second vacuum gauge 19 would then have on the display face 0 to 700 mm Hg, or the full range of the vacuum motor 12. The user would then be able to better monitor the low and mid setting on the larger vacuum gauge 18, which will subsequently have a greater viewing distance in the display and then monitor the upper vacuum levels of 300 mm Hg and above on the second gauge 19. This will also allow the user to use the device for different purposes.

Although the invention has been shown and described with respect to specific detailed embodiments thereof, it will be understood by those skilled in the art that various changes in form and detail thereof may be made without departing from the spirit and the scope of the invention.

What is claimed is:

1. An oocyte aspirator assembly, said assembly comprising:
a) means for creating a vacuum, said means comprising a vacuum pump;
b) a plurality of vacuum selectors operatively connected to said vacuum pump and operative to produce different operating vacuum pressures in a suction port component of said assembly;
c) first means for selectively connecting one or more of said vacuum selectors with said vacuum pump so as to serve to control vacuum pressures created at said suction port; and
d) second means for selectively energizing said first means.

2. The assembly of claim 1 wherein said operating vacuum pressures include a first low pressure which is in the range of about 20 mm Hg to about 120 mm Hg.

3. The assembly of claim 2 wherein said operating vacuum pressures also include a second higher pressure which is in the range of about 120 mm Hg to about 150 mm Hg.

4. The assembly of claim 3 further comprising a first pressure gauge which is operative to display operating pressures which are in the range of about 20 mm Hg to about 150 mm Hg.

5. The assembly of claim 3 further comprising a second pressure gauge which is operative to display the operating pressure which is extant during free flow of said vacuum pump.

6. The assembly of claim 3 wherein said second means includes at least one foot pedal which can be manually operated to energize said first means.

7. The assembly of claim 6 wherein said second means includes a pair of foot pedals one of which is operative to energize said first means so as to create said first low operating vacuum pressure and the other of which is operative to energize said first means so as to create said second higher operating pressure.

8. The assembly of claim 3 wherein said operating vacuum pressures also include a third high pressure which is about 350 mm Hg.

9. The assembly of claim 8 further comprising a manually operated system-occlude member on a system control panel, actuation of said member being operative to cause maximum operation of said vacuum pump and to negate operation of said first means whereby full vacuum pump pressure is created at said suction port component.
